(19) United States
(12) Patent Application Publication
(10) Pub. No.: US 2007/0037874 A1
(43) Pub. Date: Feb. 15, 2007

(54) Manufacturing process of isoflavan or isoflavene derivatives

(76) Inventors: Sang-Ku Yoo, Gyeonggi-do (KR); Ho-Kyoung Kang, Gyeonggi-do (KR); Ku-Seok Kang, Gyeonggi-do (KR); Keepyung Nahm, Seoul (KR); Sang Woo You, Seoul (KR)

Correspondence Address: JHK LAW, P.O. BOX 1078, LA CANADA, CA, 91012-1078, US

(21) Appl. No.: 10/595,410

(22) PCT Filed: Oct. 20, 2004
(86) PCT No.: PCT/KR04/02685

§ 371 Date: Apr. 14, 2006

(30) Foreign Application Priority Data
Oct. 20, 2003 (KR) 10-2003-0073168

(51) Int. Cl. A61K 31/353; C07D 311/02

(52) U.S. Cl. 514/456; 549/405

(57) ABSTRACT

The present invention relates to a method of synthesizing isoflavan and isoflavene derivatives of the Formula 1, which have the biological efficacy of antioxidation and protection against ultraviolet light. The method is effective and suitable for a mass production of isoflavan and isoflavene derivatives, which is a more convenient alternative preparation method compared to the isolation method by the extraction of plants, such as licorice, via troublesome preparative processes.

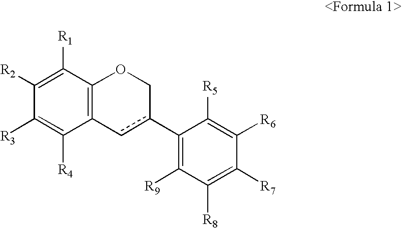

### TECHNICAL FIELD

The present invention relates to a method of synthesizing isoflavan and isoflavene derivatives of the Formula 1, which have a biological efficacy of antioxidation and protection against ultraviolet light.

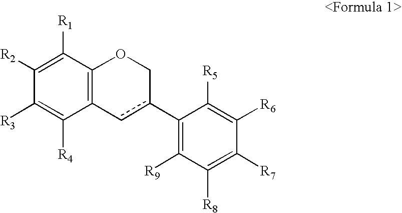

### Background Art

Flavonoids represent a large natural compound family that is widespread in the plants. Some flavonoids have many efficacies; such as activities of antibiotics, anticancer, antiviral, antiallegy, antitumor, etc. with less toxicities. According to up-to-date research, more than 3,000 flavonoids have been identified and their utilization has been paid attentions because of their biological activities. Molecular structures of flavonoids comprise one phenyl ring (A), one benzopyran ring fused to the A ring, and another phenyl ring (B) attached to the benzopyran. Flavonoids are divided into a flavonoid group and an isoflavonoid group according to connecting position of a secondary ring. The flavonoid group has a 2-phenyl ring and the isoflavonoid group has a 3-phenyl ring. They are further classified into subclasses depending on oxidation states of benzopyran rings. When the benzopyran rings are not formed but simply attached to the ring A, they are classified as a chalcone class.

The Formula 1 belongs to the isoflavonoid group. Only a few examples of the isoflavonoid group are known and the examples include isoflavans with a saturated pyran ring and isoflavene with a unsaturated pyran ring. Representative examples of isoflavans are presented in the following structural Formula and they are equol (R₁═H, R₂═H), bestitol (R₁═Me, R₂═OH), and stativan (R₁═Me, R₂═OMe). They are not found in plants, but biosynthesized from various isoflavones by intestinal microorganism of some herbivorous animals and expelled with urines from the animals.

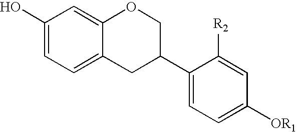

Recently, new isoflavan derivatives were discovered from licorice; Glabridin (R₁═H, R₂═H, R₃═H) and derivatives thereof, Hispaglabridin A (R₁═H, R₂═H, R₃═isoprenyl), 2′-O-Methylglabridnin (R₁═H, R₂═Me, R₃═H), 4′-O-Methylglabridnin (R₁═Me, R₂═H, R₃═H), 2′,4′-O-Dimethylglabridnin (R₁═Me, R₂═Me, R₃═H), Licoricidin, Gancanol C, etc. Isoflavene derivatives, Glabrene and Neorauflavene were also discovered from licorice, which have similar chemical structures but different biological activities compared to Glabridin. Neorauflavene may be found in other plants. 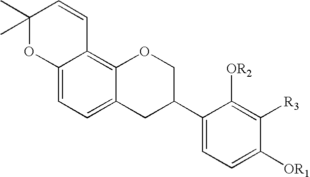
Glabridin and derivatives thereof 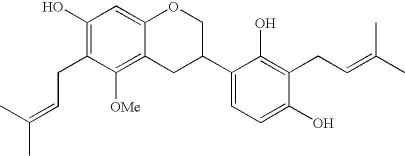
Licoricidin 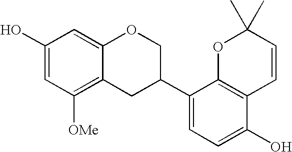
Gancanol 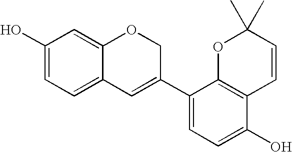
Glabrene

Licorice has been used widely for medicinal purpose, efficacy of Licorice is known to be originated from the anti-oxidative effect of isoflavan and isoflavene derivatives {Belinky, P. A., Aviram, M., Mahmood, S. and Vaya, J. (1998): structural aspects of the inhibitory effect of Glabridin on LDL Oxidation. Free. Radic. Biol. Med., 24(9), 1419-1429}. U.S. Pat. No. 4,639,466 and PCT WO 01/32191 describe that isoflavan and isoflavene derivatives are also responsible for the medicinal effect for melasma, skin cancer, osteoporosis, central nervous system (CNS) diseases, hyperpiesia, etc.

A wide range of bioactivity of isoflavan and isoflavene derivatives is known and the need for use of the compounds is increased, however the synthetic methods for the compounds are not fully developed. Only hydrogenation of isoflavone compounds may give isoflavans {Lamberton, J. A., Suares, H. and Watson, K. G. (1978): Catalytic Hydrogenation of Isoflavones. Aust. J. Chem., 31, 455-457}.

The method of hydrogenation has disclosed a synthesis of isoflavan via hydrogenation of daidzein and the derivatives extracted from plants. However, the hydrogenation condition needs high-pressure hydrogen (6,000˜10,000 kPa) with palladium catalyst, and a product is a mixture of several compounds, which prevents the method of hydrogenation to be suitable for a large scale synthesis of various derivatives of isoflavan and isoflavene containing olefinic unsaturated bonds. Up to date, derivatives of isoflavan and isoflavene are acquired only by troublesome extraction of licorice.

Several patents, JP5320152, JP6256353, DE19615576, describe the synthetic methods of isoflavan and isoflavene derivatives only from extracted glabridin as a starting material, and in JP8275792, glabridin is isolated from tissue culture. All above-mentioned methods are not appropriate for synthesis of glabridin.

### DISCLOSURE OF THE INVENTION

Technical Problem

It is an object to provide a method of synthesizing isoflavan and isoflavene derivatives of the Formula 1, which is much improved and convenient industrial production method without an extraction method from plants, such as licorice, by troublesome preparative processes.

Technical Solution

To achieve the above object, the present invention comprises three preparation steps to synthesize a compound of the Formula 1(isoflavan derivatives and isoflavene derivatives); a condensation step of a compound of the Formula 2 and a compound of the Formula 3 under basic condition to give a compound of the Formula 4; a reduction step of a compound of the Formula 4 to give a compound of the Formulas 5a and 5b; a etherification step of a compound of the Formula 5 to yield a compound of the Formula 1 (1a or 1b).

The compound of the Formula 5 includes either the compound of the Formula 5a prepared by reducing an ester group of a α-phenyl-cinnamate compound (the Formula 4) and the compound of the Formula 5b prepared by reducing an ester group and an olefinic double bond of the compound of the Formula 4.

Further, the compound of the Formula 1 includes a compound of the Formula 1a prepared by etherizing the compound of the Formula 5a and a compound of the Formula 1b prepared by etherizing the compound of the Formula 5b.

In the second step of synthesizing the compound of the Formula 5, the selective reduction of the ester group to an alcohol group of an α-phenyl-cinnamate gives the compound of the Formula 5a, and the reduction of both an ester group and a double bond gives the compound of the Formula 5b. The compound of the Formula 5a may be converted into the compound of the Formula 5b via hydrogenation.

The present invention may also comprise the suitable protection/deprotection for the above three preparation steps.

The present invention also comprises novel compounds of the Formula 4 and 5, which are important intermediates for preparing the compound of the Formula 1.

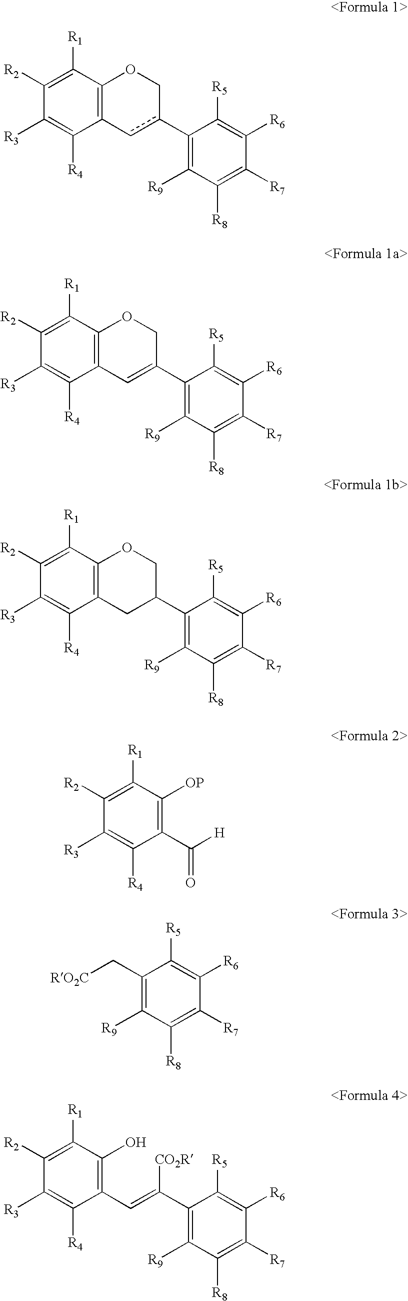

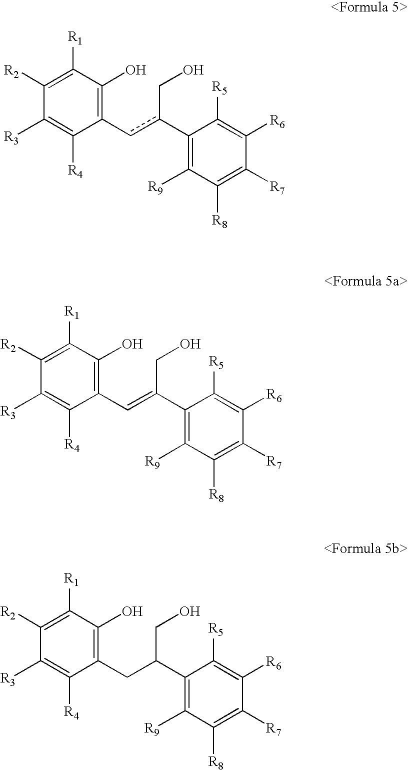

In the Formulas 1 to 5, substituents of R₁, R₂, R₃, R4, R₅, R₆, R₇, R₈ and R₉ are independent of each others and represent a hydrogen, a hydroxy, a halogen, a straight or branched alkyl group, an alkenyl group, a haloalkyl group, an alkoxy group, an alkoxyalkyl group, an alkyloxy group, an alkynyloxy group, an alkylcarbonyloxy group, an alkenylcarbonyloxy group, or an alkynylcarbonyloxy group having from 1 to 10 carbon atoms, an amine group having a general Formula of NR₁₀R₁₁, an amide group having a general Formula of R₁₀NCOR₁₁, a nitro group, a cyano group, an alkylthio group, an akenylthio group and an alkynylthio group having from 1 to 20 carbons, a phenyl group, a substituted phenyl group, a benzyl group, and a substituted benzyl group;

In the group of R₁, R₂, R₃, R₄ or R₅, R₆, R₇, R₈, R₉, any two adjacent substituents may be interlinked through —OCH₂O—, —SCH₂S—, —OCO₂—, —OCH₂CH₂O—, —OCH₂S—, —OCH₂CH₂—, —OCH₂CH₂CH₂—, —OCH₂CH═CH—, —OCMe₂CH₂CH₂—, —OCMe₂CH═CH—, —SCH₂CH₂S—, —SCH₂CH₂—, —SCH₂CH₂CH₂—, —SCH₂CH═CH—, —SCMe₂CH₂CH₂—, —SCMe₂CH₂CH₂—, —SCMe₂CH═CH—, a fused benzene ring, a furan ring, an indole ring, and a pyridin ring.

The substitituents of R′, R₁₀ or R₁₁ of the Formula 3 represent an alkyl group, an alkenyl group, an alkynyl group, a haloalkyl group and an alkoxyalkyl group having from 1 to 20 carbons.

Now, the present invention will be described in further detail in the followings.

Preparation Step 1. Condensation

The first step according to the present invention is a process of synthesizing α-phenyl-cinnamate compound of the Formula 4 by condensing the phenyl acetate compound of the Formula 3 and an O-hydroxybenzaldehyde of the Formula 2. (Reaction Formula 1)

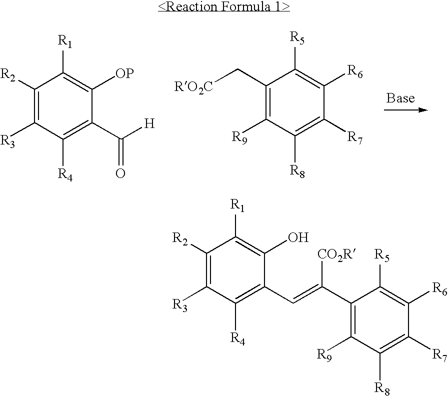

The phenyl acetate compound of the Formula 3 may be synthesized according to known methods (Carmack, M., Organic Reaction, 3, 83˜107 (1946); Carter, H. E., Organic Reaction, 3, 198˜240 (1946); Plucker, J., Amstutz, E. D., J. Am.,Chem. Soc., 62, 1512˜1513 (1940); Niederl, J. B., Ziering, A., J. Am.,Chem. Soc., 62, 885˜886 (1942); Schollkopf, V. U., Schroder, R., Angew. Chem., 85, 402˜403 (1973); McKillop, A., Swann, B., Taylor, E. C., J. Am. Chem. Soc., 95, 3340˜3343 (1973)).

An O-hydroxy group of the Formula 2 may be protected before the condensation with an appropriate protecting group, such as benzoyl chloride, pivaloyl chloride, methoxycarbonyl chloride and trimethylsilyl chloride. Protection of the O-hydroxybenzaldehyde of the present invention may increase efficiency of the condensation, reduce the amount of bases used in the condensation and improve the chemical yield.

In the condensation step, the compound of the Formula 3 is dissolved in Tetrahydrofuran(THF) or diethyl ether having a base at low temperature (<about 0° C.), which gives a corresponding enolate compound to be condensed with the protected O-hydroxybenzaldehyde compound of the Formula 2. The base may include Lithium diisopropylamide (LDA), Lithium 1,1,1,3,3,3-hexamethyl disilazide, NaNH₂, KO^{t}Bu, etc.

When a phenyl acetonitrile instead of a phenylacetate compound of the Formula 3 is used, the condensation may be performed in a mild condition, but a nitrile group has to be hydrolyzed for the next step.

Preparation Step 2. Reduction

The second step according to the present invention is a process of syntheszing the compound of the Formula 5 (either 5a or 5b) by reducing an α-phenyl-cinnamate compound of the Formula 4 prepared in the preparation step 1.
(Reaction Formula 2)

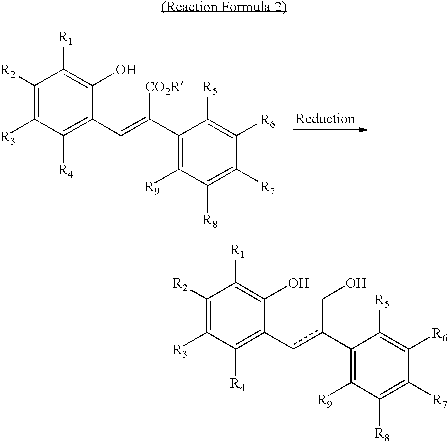

Reduction in the present invention is described in the following drawing.

The reduction in the present invention may give either the compound of the Formula 5a prepared by reducing the ester group to the alcohol group of the α-phenyl-cinnamate compound of the Formula 4, or the compound of the Formula 5b by reducing both the olefinic double bond and the ester group or by reducing the double bond and then reducing the ester group to the alcohol, and the compound of the Formula 5a may be converted to the compound of the Formula 5b by conventional hydrogenation methods.

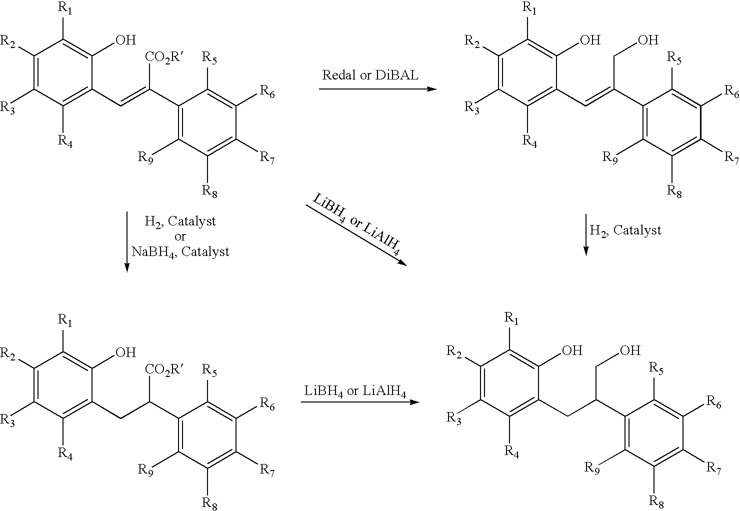

The reduction of the ester group only to the alcohol of the α-phenyl-cinnamate compound of the Formula 4 in the present invention needs reducing agents, for examples, DIBAL, KBH(CHMeEt), LiBH(CHMeEt)₃, NaAlH₂(OCH₂CH₂OMe)₂, LiAlH₂(OEt)₂, etc. to give the compound of the Formula 5a.

The reduction of both the double bond and the ester group of the compound of the Formula 4 and the reduction of the ester group of the compound of the Formula 6 compound may be performed with an reducing agent selected from the group consisting of LiAlH₄, NaAlH₄, LiBH₄, LiBEt₃, etc. to give the compound of the Formula 5b.

The reduction of the olefinic double bond of the compound of the Formula 4 is performed in conditions using one selected from the group consisting of NaBH₄, LiBH₄, etc. with Lewis acid catalyst or hydrogenating with Nickel, Palladium, Platinum, Ruthenium, Rhodium, etc. as a catalyst, and the reduction of the olefinic double bond of the compound of the Formula 5a also needs the hydrogenation to afford a compound of the Formula 5b. Especially, the hydrogenation of the olefinic double bond with a chiral catalyst may induce the stereo-selective hydrogenation at the 3-position of the isoflavan.

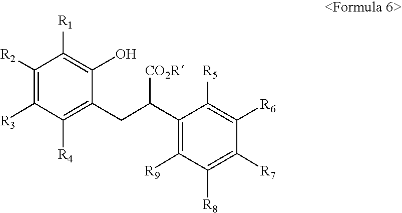

In the Formula 6, substituents of R₁, R₂, R₃, R4, R₅, R₆, R₇, R₈, R₉ and R′ are as defined in the above.

Preparation Step 3. Etherification

The third step according to the present invention is a process for synthesizing the compound of the Formula 1 described as the Formulas 1a and 1b by etherizing and cyclizing the compound of the Formula 5 prepared in the preparation step 2 via an ether bond. (Reaction Formula 3)

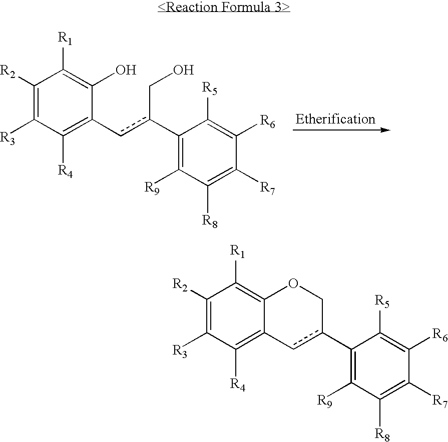

The etherification of the present invention is performed by the known Mitsunobu reaction with diethylazodicarboxylate (DEAD) and triphenyl phosphin, or by synthesizing mesylate or tosylate of the primary alcohol of the compound of the Formula 5, which is then cyclized with a base of NaOH, KOH, etc.

Advantageous Effects

As described above, the present invention provides an method of preparing an isoflavan derivative and an isoflavene derivative of the Formula 1, including a preparation step 1 for the synthesis of a compound of the Formula 4 prepared by condensing a compound of the Formula 2 and a compound of the Formula 3 in the presence of a base; a preparation step 2 for the synthesis of a compound of the Formula 5, more precisely the Formula 5a or the Formula 5b, by reducing a compound of the Formula 4; a preparation step 3 for the synthesis of a compound of the Formula 1, more precisely the Formula 1a or the Formula 1b, by etherizing a compound of the Formula 5. The method of the present invention is more effective and convenient in the production of an isoflavan derivative or isoflavene derivative than the extraction method with licorice, and provides a way to the mass production of the derivatives of antioxidative and UV-screening efficacy.

### BEST MODE FOR CARRYING OUT THE INVENTION

The preparation examples and examples are given solely for the purpose of illustration and are not to be construed as limitations of the present invention, as many variations thereof are possible without departing from the spirit and scope of the invention.

### PREPARATION EXAMPLE 1

### Preparation of 5-benzoyloxy-2,2-dimethyl-6-formyl-2H-1-benszopyran

2,2-dimethyl-6-formyl-5-hydroxy-2H-1-benzopyran (2.04 gr, 10.0 mmol) synthesized according to the reference (Clarke, D., Crombie, L., Whiting, D. A., J.Chem., Chem. Comm., 1973, 580p-582p), benzoyl chloride (1.48 gr, 10.5 mmol) and K₂CO₃ (1.38 gr, 10.0 mmol) were dissolved in acetone (30 mL) and stirred for 3 hours. The solution was filtered to remove salt, the filterate was concentrated under reduced pressure. The residue was dissolved in ethyl acetate and washed with brine, then dried over anhydrous MgSO₄ and concentrated under reduced pressure to give 5-benzoyloxy-2,2-dimethyl-6-Formyl-2H-1-benzopyran (3.08 gr, 10.0 mmol).

¹H-NMR(CDCl₃): 9.92(s, 1H), 8.25(d, 2H), 7.71(d, 2H), 7.70(t, 1H), 7.55(t, 2H), 6.83(d, 1H), 6.38(d, 1H), 5.69(d, 1H), 1.49(s, 6H)

### PREPARATION EXAMPLE 2

### Preparation of 2,2-dimethyl-6-Formyl-5-pivaroyloxy-2H-1-benzopyran

5-benzoyloxy-2,2-dimethyl-6-Formyl-2H-1-benzopyran (2.04 gr, 10.0 mmol) and pivaloyl chloride (1.3gr, 10.5 mmol) were dissolved in acetone (30 mL). 2,2-dimethyl-6-Formyl-5-pivaroyloxy-2H-1-benzopyran was obtained (2.88 gr, 10.0 mmol) as described in the Preparation example 1.

¹H-NMR(CDCl₃): 9.85(s, 1H), 7.65(d, 1H), 6.77(d, 1H), 6.29(d, 1H), 5.71(d, 1H), 1.47(s, 6H), 1.44(s, 9H)

### PREPARATION EXAMPLE 3

### Preparation of methyl 2′,4′-dibenzyloxyphenylacetate

2′,4′-dibenzyloxyacetophenone (3.32 gr, 10.0 mmol) was dissolved in methanol (50 mL), then hyperchloric acid (5 mL) was added. Ti(NO₃)₃·3H₂O (5.55 g, 12.5 mmol)was added slowly to the solution over 30 minutes and the solution was stirred for 5 hours at room temperature. The solution was filtered and concentrated. The residue was dissolved in ethyl acetate (50 mL) and washed with brine twice (2×50 mL), then dried over anhydrous MgSO₄ and concentrated under reduced pressure to give methyl 2,4-dibenzyloxyphenylacetate (3.15 g, 8.7 mmol).

¹H-NMR(CDCl₃): 7.3˜7.5(b, 10H), 7.11(d, 1H), 6.60(d, 1H), 6.54(dd, 1H), 5.03(s, 4H), 3.63(s, 3H), 3.61(s, 2H)

### PREPARATION EXAMPLE 4

### Preparation of methyl 2′,4′-dimethoxyphenylacetate

Methyl 2′,4′-dimethoxyphenylacetate was synthesized with 2′,4′-methoxyacetophenone (9.0 g, 50 mmol) in methanol 80 mL as described in the Preparation example 3.

¹H-NMR(CDCl₃): 7.3˜7.5(b, 10H), 7.11(d, 1H), 6.60(d, 1H), 6.54(d, 1H), 5.03(s, 4H), 3.63(s, 3H), 3.61(s, 2H)

### PREPARATION EXAMPLE 5

### Preparation of methyl 2′,4′-di(methoxymethoxy)phenylacetate

To the mixture of 2′,4′-dihyroxyacetophenone (7.61 g, 50.0 mmol) and diisopropylethylamine 14.2 g(110 mmol) was added methoxymethylchloride (8.85 g, 110 mmol) with stirring at an ice-water bath for 30 minutes. The solution was further stirred at room temperature. Sodium hydroxide aqueous solution (20 mL, NaOH 4.4 g, 0.12 mmol) was added to the reaction solution for 30 minutes, then the organic phase was separated and distilled under reduced pressure to give 2′,4′-di(methoxymethoxy)acetophenone (10.9 g, 45.4 mmol, b.p.: 145˜160° C. /0.4 mmHg). Methyl 2′,4′-di(methoxymethoxy)phenylacetate was synthesized from 2′,4′-di(methoxymethoxy)acetophenone as described in the Preparation example 3.

¹H-NMR(CDCl₃): 7.09(d, 1H), 6.80(d, 1H), 6.67(dd, 1H), 5.17(s, 2H), 5.15(s, 2H), 3.68(s, 3H), 3.59(s, 2H), 3.47(s, 3H), 3.45(s, 3H)

### PREPARATION EXAMPLE 6

### Preparation of 2,2-dimethyl-6-formyl-5-hydroxy dihydrobenzypyran

2,2-Dimethyl-6-formyl-5-hydroxy-2H-1-benzopyran (2.04 gr, 10.0 mmol) synthesized according to the reference (Clarke, D., Crombie, L., Whiting, D. A., J.Chem., Chem. Comm., 1973, 580p-582p) was dissolved in methanol (15 mL) containing 5% Pd/C (50 mg). The solution was sealed with a hydrogen balloon and saturated with hydrogen gas and the solution was stirred for 10 hours. The solution was filtered and the filterate was concentrated to give 2,2-dimethyl-6-formyl-5-hydroxydihydrobenzypyran (2.06 g, 10.0 mmol).

¹H-NMR(CDCl₃): 9.65(s, 1H), 7.27(d, 1H), 6.43(d, 1H), 2.69(t, 2H), 1.83(t, 3H), 1.36(s, 6H)

### EXAMPLE 1

### Preparation of 2′,4′-dibenzylgrabridin

The First Step:

To THF solution of LDA (1.0M, 12 mL) cooled to −78° C. in dry ice-acetone bath was added THF (5 mL) solution of methyl 2,4-dibenzyloxyphenylacetate (3.62 g, 10.0 mmol) for 10 minutes with stirring, then the solution of 5-benzoyloxy-2,2-dimethyl-6-Formyl-2H-1-benzopyran (3.08 g, 10.0 mmol)in 5 mL THF was slowly added for 10 minutes and further stirred for 30 minutes, then brine (100 mL) was added. The solution was stirred for 30 minutes and the organic layer was separated and the aqueous layer was extracted with ethyl acetate (50 mL). The combined organic layer was dried over MgSO4, and concentrated. The residue was purified by column chromatography to give 2-(2,4-dibenzyloxyphenyl)-3-(2,2-dimethyl-5-hydroxy-2H-1-benzypyran-6-yl) acrylic acid methyl ester (4.85 g, 8.85 mmol).

¹H-NMR(CDCl₃): 7.81(s, 1H), 7.2˜7.5(b, 10H), 6.94(d, 1H), 6.70(d, 1H), 6.63(s, 1H), 6.56(d, 1H), 6.50(d, 1H), 5.54(d, 1H), 5.00(s, 4H), 3.70(s, 3H), 1.39(s, 6H). ¹³C-NMR(CDCl₃): 171.56, 160,18, 157.30, 154.72, 150.27, 136.63, 135.64, 133.65, 131.77, 130.15, 128.73, 128.56, 128.43, 128.05, 127.75, 127.65, 127.57, 127.00, 117.61, 116.55, 114.97, 109.54, 109.08, 106.23, 105.78, 100.94, 76.15, 70.13, 52.26, 27.87. Mass (ApCI): 549(M⁺¹), 517

The Second Step:

To THF solution (20 mL) of methyl 2-(2,4-dibenzyloxyphenyl) -3-(2,2-dimethyl-5-hydroxy-2H-1-benzypyran-6-yl) acrylate (2.74 g, 5.0 mmol) was added the THF solution (15 mL) of LiBH₄ (1.0 M) and the solution was refluxed for 5 hours with stirring. The solution was cooled in an ice-water bath, and 20 mL of 1N HCl aqueous solution was added slowly, and then extracted with ethyl acetate (50 mL). The organic layer was dried over MgSO₄, concentrated under reduced pressure, and then was purified by column chromatography to give 2-(2,4-dibenzyloxyphenyl)-3-(2,3-dimethyl-5-hydroxy--2H-1-benzopyran-6-yl) propan-1-ol (1.22 g, 2.34 mmol).

¹H-NMR(CDCl₃): 7.2˜7.5(b, 10H), 7.15(d, 1H), 6.72(d, 1H), 6.67(m, 2H), 6.30(d, 1H), 5.55(d, 1H), 5.06(s, 2H), 5.04(s, 2H), 3.81(dd, 1H), 3.70(dd, 1H), 3.28(m, 1H), 3.08(dd, 1H), 2.67(dd, 1H), 1.42(s, 3H), 1.40(s, 3H) ¹³C-NMR(CDCl₃): 158.65, 156.72, 152.35, 150.94, 136.81, 136.21, 130.73, 128.78, 128.71, 128.59, 128.23, 128.02, 127.56, 127.52, 127.20, 123.94, 117.99, 117.55, 110.24, 108.41, 105.59, 100.96, 75.47, 70.45, 70.15, 63.39, 41.89, 30.50, 27.87, 27.56. Mass (ApCI): 523(M⁺¹), 505 m.p.: 63˜65° C.

The Third Step:

To THF solution (10 mL) of 2-(2,4-dibenzyloxyphenyl)-3-(2,3-dimethyl-5-hydroxy--2H-1-benzopyran-6-yl) propan-1-ol (1.22 g, 2.34 mmol) was added triphenylphosphine (0.919 g, 3.51 mmol), then a toluene solution of diethylazodicarboxylate (1.0 M, 3.0 mL) was added slowly and the solution was stirred for 1 hour at ambient temperature. The solution was concentrated and purified by column chromatography to give 2′,4′-dibenzylglabridin (0.97 g, 1.9 mmol).

The NMR spectra of the above 2′,4′-dibenzylglabridin is exactly matched with that of 2′,4′-dibenzylglabridin that was synthesized from natural extracted glabridin and benzoyl chloride.

¹H-NMR(CDCl₃): 7.2˜7.5(b, 10H), 7.03(d, 1H), 6.81(d, 1H), 6.64(d, 1H), 6.62(s, 1H), 6.54(d, 1H), 6.36(d, 1H), 5.55(d, 1H), 5.06(s, 2H), 5.01(s, 2H), 4.36(dd, 1H), 4.02(dd, 1H), 3.67(m, 1H), 2.92(dd, 1H), 2.80(dd, 1H), 1.42(s, 3H), 1.40(s, 3H). ¹³C-NMR(CDCl₃): 158.68, 157.22, 151.79, 149.79, 136.87, 136.78, 129.13, 128.78, 128.57, 127.98, 127.86, 127.68, 127.48, 127.09, 122.54, 116.94, 114.40, 109.81, 108.55, 105.62, 100.74, 75.51, 70.12, 70.05, 31.29, 30.67, 29.65, 27.75, 27.54. Mass (ApCI): 505(M⁺¹)

### EXAMPLE 2

### Preparation of 2′,4′-dimethylgrabridin

The First Step:

Methyl 2′,4′-dimethoxylacetate (2.10 g, 10.0 mmol) acquired from the Preparation example 4 and 2,2-dimethyl-6-Formyl-5-pivaroyloxy-2H-1-benzopyran (2.88 g, 10.0 mmol) from the Example 2 were treated as described in Example 1 to give methyl 2-(2,4-dimethoxyphenyl)-3-(2,2-dimethyl -5-hydroxy-2H-1-benzopyran-6-yl)acrylate (3.61 g, 9.1 mmol).

¹H-NMR(CDCl₃): 7.83(s, 1H), 6.90(d, 1H), 6.69(d, 1H), 6.57(d, 1H), 6.40(dd, 1H), 6.20(d, 1H), 5.52(d, 1H), 3.80(s, 3H), 3.75(s, 3H), 3.74(s, 3H), 1.38(s, 6H). ¹³C-NMR(CDCl₃): 169.23, 160,87, 158.24, 154.57, 150.65, 142.07, 135.67, 131.42, 129.91, 128.57, 127.79, 117.27, 116.49, 115.15, 109.48, 108.82, 104.85, 98.83, 75.96, 55.48, 55.15, 27.72. Mass (ApCI): 397(M⁺¹), 365 m.p.: 82˜84° C.

The Second Step:

To 1,4-dioxane solution (35 mL) of Methyl 2-(2,4-dimethoxyphenyl)-3-(2,2-dimethyl-5-hydroxy-2H-1-benzopyran-6-yl)acrylate (3.61 g, 9.1 mmol) was added 10 mL THF solution of 1.0 M LiBH₄. The solution was stirred for 5 hours at ambient temperature, cooled in an ice-water bath, added 1 N aqueous HCl (20 mL) slowly, and extracted with ethyl acetate (50 mL). The organic layer was dried (MgSO4), and concentrated under reduced pressure. The crude residue was purified by chromatography on silica gel to give methyl 2-(2,4-dimethoxyphenyl)-3-(2,3-dimethyl-5-hydroxy-2H-1-benzopyran-6-yl) propanoate (2.26 g, 5.7 mmol).

¹H-NMR(CDCl₃): 7.83(s, 1H),7.00(d, 1H), 6.78(d, 1H), 6.73(d, 1H), 6.47(s, 1H), 6.46(d, 1H), 6.30(d, 1H), 5.57(d, 1H), 4.11(dd, 1H), 3.80(s, 6H), 3.65(s, 3H), 3.16(dd, 1H), 3.28(dd, 1H), 1.42(s, 3H), 1.40(s, 3H). ¹³C-NMR(CDCl₃): 177.44, 160,14, 157.17, 152.50, 150.34, 130.71, 128.59, 128.30, 120.38, 118.13, 117.58, 110.65, 108.53, 104.50, 98.97, 75.50, 55.55, 55.34, 52.61, 47.06, 32.82, 27.85, 27.61. Mass (ApCI): 399(M⁺¹), 367, 339 m.p.: 64˜67° C.

To cooled THF solution (10 mL) of Methyl 2-(2,4-dimethoxyphenyl)-3-(2,3-dimethyl-5-hydroxy-2H-1-benzopyran-6-yl) propanoate (2.26 g, 5.7 mmol) was added LiAlH₄ (0.24 g, 6.0 mmol). The solution was refluxed for 1 hour with stirring, added in 0.3 mL of water, stirred for 5 minutes with stirring, added in 0.3 mL of aqueous 15% NaOH, stirred for 10 minutes, and added again in 10 mL of water. The mixture was filtered and the organic layer was separated, dried (MgSO₄), and concentrated under reduced pressure. The crude residue was purified by chromatography on silica gel to give 2-(2,4-dimethoxyphenyl)-3-(2,3-dimethyl-5-hydroxy-2H-1-benzopyran-6-yl) propan-1-ol (1.44 g, 3.9 mmol).

¹H-NMR(CDCl₃): 7.15(d, 1H), 6.78(d, 1H), 6.74(d, 1H), 6.52(d, 1H), 6.48(dd, 1H), 6.32(d, 1H), 5.57(d, 1H), 3.86(s, 3H), 3.81(s, 3H), 3.78(m, 2H), 3.22(m, 1H), 3.01(dd, 1H), 2.67(dd, 1H), 1.43(s, 3H), 1.41(s, 3H). ¹³C-NMR(CDCl₃): 159,60, 157.61, 152.44, 150.93, 130.69, 128.76, 128.46, 123.31, 118.06, 117.55, 110.28, 108.42, 104.28, 99.07, 75.53, 63.32, 55.53, 55.39, 41.74, 30.90, 27.83, 27.63. Mass (ApCI): 371(M⁺¹), 353 m.p.: 103˜104° C.

The Third Step:

To THF solution (20 mL) of NaH(50%) (0.50 g, 10.0 mmol) was slowly added a THF solution of 2-(2,4-dimethoxyphenyl)-3-(2,3-dimethyl-5-hydroxy-2H-1-benzopyran-6-yl) propan-1-ol (1.44 g, 3.9 mmol) and p-Toluenesulfonyl chloride (0.82 g, 4.3 mmol). The solution was stirred at ambient temperature for 1 hour, and then refluxed for 2 hours. The solution was extracted and purified by chromatography on silica gel to give 2′,4′-dimethylglabridin (0.953 g, 2.7 mmol).

The NMR spectra of the above 2′,4′-dimethylglabridin is exactly matched with that of 2′,4′-dimethylglabridin which was synthesized from natural extracted glabridin and dimethylsulfate.

¹H-NMR(CDCl₃): 7.02(d, 1H), 6.82(d, 1H), 6.65(d, 1H), 6.48(s, 1H), 6.45(d, 1H), 6.36(d, 1H), 5.55(d, 1H), 4.34(dd, 1H), 3.98(t, 1H), 3.80(s, 6H), 3.56(m, 1H), 2.96(dd, 1H), 2.82(dd, 1H), 1.43(s, 3H), 1.41(s, 3H). ¹³C-NMR(CDCl₃): 159,64, 158.27, 151.81, 149.77, 129.15, 128.82, 127.52, 121.85, 116.97, 114.51, 109.84, 108.55, 104.09, 98.67, 75.50, 70.19, 55.32, 55.30, 31.47, 30.58, 27.76, 27.48. Mass (ApCI): 353(M⁺¹) m.p.: 97˜98° C.

### EXAMPLE 3

### Preparation of 2′,4′-di(methoxymethyl)grabridin and glabridin

The First Step:

Methyl 2′,4′-di(methoxymethyl)phenylacetate prepared in Preparation Example 5 was treated as described in Example 1 to give methyl 2-(2′,4′-di(methoxymethoxy)phenyl)-3-(2,2-dimethyl-5-hydroxy -2H-1-benzopyran-6-yl)acrylate (3.46 g, 7.6 mmol).

¹H-NMR(CDCl₃): 7.81(s, 1H), 6.90(d, 1H), 6.86(d, 1H), 6.71(d, 1H), 6.61(dd, 1H), 6.53(d, 1H), 6.22(d, 1H), 5.53(d, 1H), 5.16(s, 2H), 5.08(s, 2H), 3.76(s, 3H), 3.49(s, 3H), 3.38(s, 3H), 1.39(s, 6H). ¹³C-NMR(CDCl₃): 169.03, 158,55, 155.97, 154.78, 150.49, 135.81, 131.53, 130.12, 128.80, 128.03, 119.21, 116.40, 114.91, 109.46 109.39, 109.07, 104.00, 94.89, 94.52, 76.10, 56.15, 56.01, 52.26, 27.82. Mass (ApCI): 457(M⁺¹), 425, 393 m.p.: 119˜122° C.

The Second Step:

Methyl 2-(2′,4′-di(methoxymethoxy)phenyl)-3-(2,2-dimethyl-5-hydroxy -2H-1-benzopyran-6-yl)acrylate (3.46 g, 7.6 mmol) was treated as described in Example 1 to give 2-(2′,4′-di(methoxymethoxy)phenyl)-3-(2,2-dimethyl-5-hydroxy -2H-1-benzopyran-6-yl)propan-1-ol (1.41 g, 3.27 mmol).

¹H-NMR(CDCl₃): 7.66(b, 1H), 7.16(d, 1H), 6.84(d, 1H), 6.79(d, 1H), 6.72(d, 1H), 6.68(dd, 1H), 6.32(d, 1H), 5.20(s, 2H), 5.15(s, 2H), 3.78(b, 2H), 3.47(s, 6H), 3.29(m, 1H), 3.02(dd, 1H), 2.70(dd, 1H), 1.42(s,3H), 1.41(s, 3H). ¹³C-NMR(CDCl₃): 159,95, 155.22, 152.43, 150.84, 130.61, 128.78, 128.44, 124.94, 117.92, 117.46, 110.26, 108.82, 104.46, 103.58, 94.67, 94.51, 75.50, 63.43, 56.36, 56.04, 41.29, 30.81, 27.80, 27.57. Mass (ApCI): 431(M⁺¹), 399, 381

The Third Step:

To THF solution (10 mL) of 2-(2′,4′-di(methoxymethoxy)phenyl)-3-(2,2-dimethyl-5-hydroxy -2H-1-benzopyran-6-yl)propan-1-ol (1.41 g, 3.27 mmol) was added triphenylphosphine (0.919 g, 3.51 mmol) and diethylazodicarboxlate (DEAD) (3.5 mL of 1.0 M toluene solution). The solution was stirred at ambient temperature for 1 hour. The solution was concentrated, and purified by chromatography on silica gel to give 2′,4′-di(methoxymethyl)glabridin (1.10 g, 2.68 mmol).

¹H-NMR(CDCl₃): 7.03(d, 1H), 6.84(s, 1H), 6.83(d, 1H), 6.68(d, 1H), 6.65(dd, 1H), 6.36(d, 1H), 5.56(d, 1H), 5.20(s, 2H), 5.15(s, 2H), 4.36(dd, 1H), 4.00(t, 1H), 3.6(m, 1H), 3.48(s, 6H), 2.97(dd, 1H), 2.84(dd, 1H), 1.43(s, 3H), 1.41(s, 3H). ¹³C-NMR(CDCl₃): 157,05, 155.83, 151.88, 149.71, 129.16, 128.94, 127.66, 123.54, 116.90, 114.39, 109.87, 108.86, 108.65, 103.46, 94.54, 94.46, 75.55, 70.19, 56.21, 56.06, 31.64, 30.76, 27.78, 27.49. Mass (ApCI): 413(M⁺¹), 381 m.p.: 74˜75° C.

The Fourth Step:

To isopropanol solution (5 mL) of 2′,4′-di(methoxymethyl)glabridin (0.412 g, 1.0 mmol) was added 0.1 mL of concentrated aqueous HCl. The solution was stirred at room temperature for 5 hours, concentrated under reduce pressure, and purified by chromatography on silica gel to give glabridin (0.265 g, 0.82 mol), whose NMR spetrum is matched exactly with that of the extracted glabridin from licorice.

¹H-NMR(CDCl₃): 6.94(d, 1H), 6.82(d, 1H), 6.65(d, 1H), 6.38(dd, 1H), 6.37(d, 1H), 6.31(d, 1H), 5.56(d, 1H), 5.20(b, 1H), 4.37(dd, 1H), 4.02(t, 1H), 3.48(m, 1H), 2.84(dd, 1H), 1.43(s, 3H), 1.41(s, 3H). ¹³C-NMR(CDCl₃): 155,25, 154.44, 151.91, 149.75, 129.18, 128.95, 128.41, 120.01, 116.95, 114.32, 109.93, 108.73, 107.98, 103.11, 75.62, 70.00, 31.70, 30.61, 27.79, 27.55. Mass (ApCI): 325(M⁺¹)

### EXAMPLE 4

### Preparation of 2′,4′-dibenzyldihydrograbridin

2,2-Dimethyl-6-formyl-5-hydroxydihydrobenzypyran prepared in Preparation example 6 was converted to 5-benzoyloxy-2,2-dimethyl-6-formyl-2H-1-dihydrobenzypyran as described in Preparation example 1, which was treated with methyl (2,4-dibenzyloxyphenyl)acetate as described in Example 1 to give 2′,4′-dibenzyldihydrograbridin.

¹H-NMR(CDCl₃): 7.30˜7.45(m. 10H), 7.04(d, 1H), 6.83(d, 1H), 6.63(d, 1H), 6.56(dd, 1H), 6.38(d, 1H), 5.07(s, 2H), 5.02(s, 2H), 4.38(dd, 1H), 4.01(t, 1H), 3.63(m, 1H), 2.98(dd, 1H), 2.87(dd, 1H), 2.63(t, 2H), 1.77(t, 2H), 1.33(s, 3H), 1.32(s, 3H).

1. A method of preparing isoflavan or isoflavene derivatives of Formula 1, comprising,
a preparation step 1 of synthesizing a compound of Formula 4 by condensing a compound of Formula 2 and a compound of Formula 3 in a base;
a preparation step 2 of synthesizing of a compound of Formula 5, including Formula 5a and Formula 5b, by reducing a compound of Formula 4; and
a preparation step 3 of synthesizing a compound of Formula 1 including Formula 1a and Formula 1b, by etherizing the compound of Formula 5. 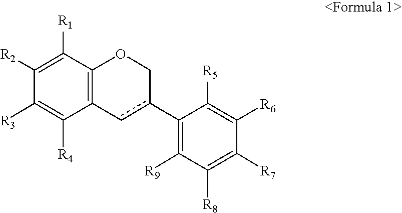 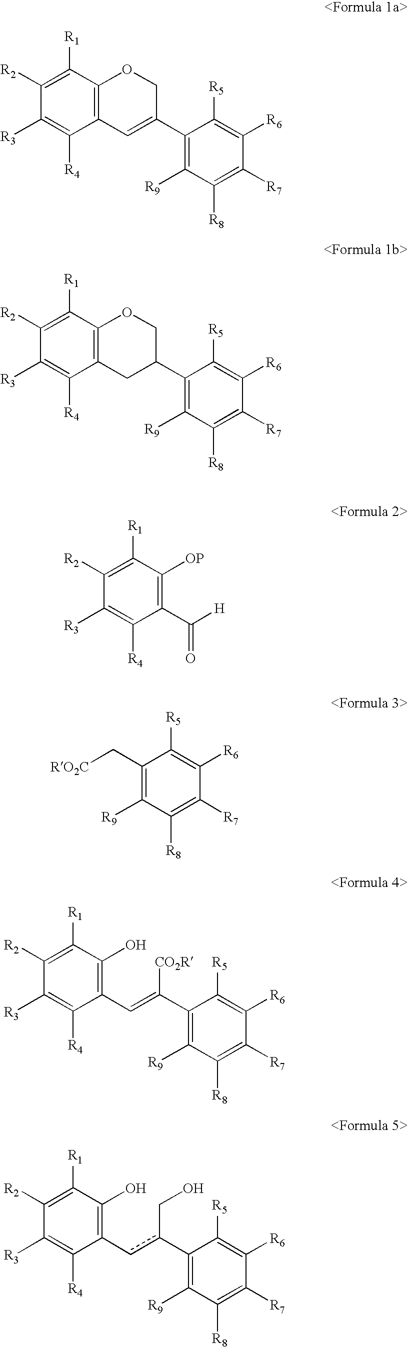 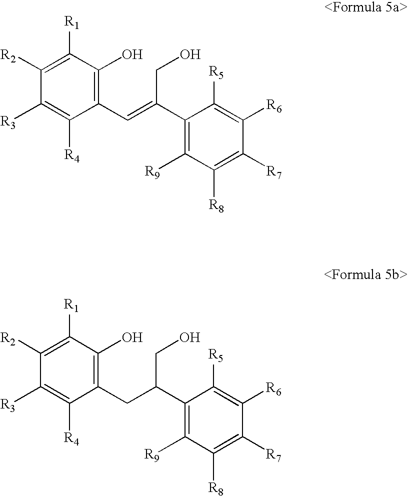
In the Formulas 1 to 5, substituents of R₁, R₂, R₃, R₄, R₅, R₆, R₇, R₈ and R₉ are independent of each others and represent a hydrogen, a hydroxy, a halogen, a straight or branched alkyl group, an alkenyl group, a haloalkyl group, an alkoxy group, an alkoxyalkyl group, an alkyloxy group, an alkynyloxy group, an alkylcarbonyloxy group, an alkenylcarbonyloxy group, or an alkynylcarbonyloxy group having from 1 to 10 carbon atoms, an amine group having a general Formula of NR₁₀R₁₁, an amide group having a general Formula of R₁₀NCOR₁₁, a nitro group, a cyano group, an alkylthio group, an akenylthio group and an alkynylthio group having from 1 to 20 carbons, a phenyl group, a substituted phenyl group, a benzyl group, and a substituted benzyl group;
In the groups of R₁, R₂, R₃, R₄, R₅, R₆, R₇, R₈ and R₉, any two adjacent substituents are interlinked through —OCH₂O—, —SCH₂S—, —OCO₂—, —OCH₂CH₂O—, —OCH₂S—, —OCH₂CH₂—, —OCH₂CH₂CH₂—, —OCH₂CH═CH—, —OCMe₂CH₂CH₂—, —OCMe₂CH═CH—, —SCH₂CH₂S—, —SCH₂CH₂—, —SCH₂CH₂CH₂—, —SCH₂CH═CH—, —SCMe₂CH₂CH₂—, —SCMe₂CH₂CH₂—, —SCMe₂CH═CH—, a fused benzene ring, a furan ring, an indole ring, or a pyridin ring.
The substitituents of R′ , R₁₀ or R₁₁ of the Formula 3 represent an alkyl group, an alkenyl group, an alkynyl group, an haloalkyl group, or an alkoxyalkyl group having 1 to 20 carbons.

2. The method of claim 1, wherein the protected o-hydroxybenzaldehyde compound of the Formula 2 is a compound protected by using one selected from the group consisting of benzoyl chloride, pivaloyl chloride, methoxycarbonyl chloride, and trimethylsilyl chloride.

3. The method of any one of claims 1 and 2, wherein a base of the preparation step 1 is one selected from the group consisting of Lithium Diisopropylamide (LDA), NaNH₂, and KO^{t}Bu.

4. The method of claim 3, wherein a reaction temperature is below about 0° C.

5. The method of claim 1, wherein a reducing agent of the preparation step 2 is one selected from the group consisting of DIBAL, KBH (CHMeEt), LiBH(CHMeEt)₃, NaAlH₂(OCH₂CH₂OMe)₂, and LiAlH₂(OEt)₂ to give a compound of the Formula 5a by reducing only the ester group of the α-phenyl-cinnamate compound of the Formula 4 for synthesizing the compound of the Formula 1a.

6. The method of claim 5, wherein the reduction of the compound of the Formula 5a to a compound of the Formula 5b is hydrogenation catalyzed by one selected from the group consisting of Nickel, Palladium, Platinum, Ruthenium and Rhodium for synthesizing the compound of the Formula 1b.

7. The method of claim 1, wherein a reducing agent of the preparation step 2 is one selected from the group consisting of LiAlH₄, NaAlH₄, LiBH₄, and LiBEt₃ to give the compound of the Formula 5b by reducing both the ester group and the olefinic double bond of the α-phenyl-cinnamate compound of the Formula 4 for synthesizing the compound of the Formula 1b.

8. The method of claim 1, wherein the reduction of the olefinic double bond of the compound of Formula 4 in the preparation step 2 is carried out by using a double bond reducing agent of one selected from the group consisting of NaBH₄ and LiBH₄ in a condition with a Lewis acid catalyst, or by hydrogenating with one selected from the group consisting of Nickel, Palladium, Platinum, Ruthenium, and Rhodium as a catalyst to give a compound of Formula 6 and then the ester group of the Formula 4 is reduced with a reducing agent selected from the group consisting of LiAlH₄, NaAlH₄, LiBH₄, and LiBEt₃ to give the compound of the Formula 5b for synthesizing the compound of the Formula 1b. 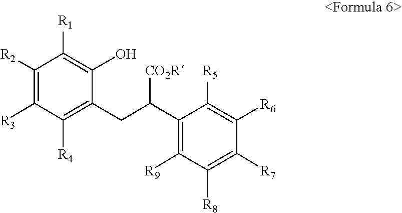
wherein, substituents of R₁, R₂, R₃, R₄, R₅, R₆, R₇, R₈ and R₉ are as defined in claim 1.

9. A compound of the Formula 4, wherein substituents of R₁, R₂, R₃, R₄, R₅, R₆, R₇, R₈, R₉ and R′ are as defined in claim 1.

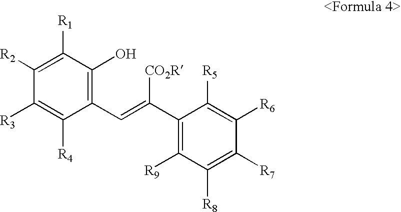

10. A compound of the Formula 5, wherein substituents of R₁, R₂, R₃, R₄, R₅, R₆, R₇, R₈, R₉ and R′ are as defined in claim 1. [chemical structure]
